(19) United States
(12) Patent Application Publication
(10) Pub. No.: US 2002/0072492 A1
(43) Pub. Date: Jun. 13, 2002

(54) Non-genetic based protein disease markers

(76) Inventors: Timothy G. Myers, Kensington, MD (US); Rembert Pieper, Washington, DC (US); John Taylor JR., Clayton, NC (US); Sandra Steiner, Gaithersburg, MD (US); N. Leigh Anderson, Washington, DC (US)

Correspondence Address: Dean H. Nakamura, Roylance Abrams Berdo & Goodman, 1300 19th Street, N.W., Washington, DC, 20036, US

(21) Appl. No.: 09/886,271

(22) Filed: Jun. 22, 2001

Related U.S. Application Data
(63) Continuation-in-part of application No. 09/660,242, filed on Sep. 12, 2000.

(51) Int. Cl. A61K 38/17; G01N 33/53; G01N 33/537; G01N 33/543

(52) U.S. Cl. 514/12; 435/7.92

(57) ABSTRACT

Protein disease markers for obesity, osteoporosis, diabetes, osteoarthritis and hypertension are disclosed. These markers are not inherited or of genetic origin as they were not found in identical twins of the affected individual. Methods and uses for diagnostic, therapeutic and drug discovery are disclosed.

### FIELD OF THE INVENTION

The instant invention relates the discovery of protein markers for diseases that do not have an apparent genetic component.

### BACKGROUND OF THE INVENTION

The studies of identical twins have allowed one to study non-genetic factors without concern for polymorphisms and mutations as the twins originated from the same zygote and thus are genetically identical. While post separation genetic mutations may occur, these are relatively few compared to the large number of differences between fraternal twins or unrelated individuals.

When examining many proteins by two dimensional electrophoresis gels from monozygotic twins and unrelated individuals, the twins always gave identical protein displays for PHA stimulated lymphocytes, Goldman et al., American Journal of Human Genetics 35(5):827-37 (1983). The same results occurred when detecting polymorphisms in serum proteins, Borresen et al., Clinical Genetics 20(6):438-48 (1981). Using two dimensional electrophoresis gels to determine protein differences for Huntington's disease also resulted in no characteristic protein on the gel.

Monozygotic twins were shown to correlate for serum carboxyl terminal propeptide of type I procollagen (PICP), serum pyridinoline crosslinked carboxyterminal telopeptide of type I collagen (ICTP), and serum aminoterminal propeptide of type III procollagen. Tokita et al., Journal of Clinical Endocrinology & Metabolism 78(6):1461-1466 (1994).

Monozygotic twins correlated with each other for various serum proteins in twin pairs that developed diabetes versus those twin pairs that did not. Hussain et al., Diabetologia 39(1):60-69 (1996) and Roder et al., Journal of Clinical Endocrinology and Metabolism 80(8):2359-63 (1995). Likewise for hypertension, McCaffery et al., Journal of Hypertension 17(12):1677-85 (Dec. 1999) (not prior art) and Robertson et al., American Journal of the Medical Sciences 318(5):298-303 (1999) (not prior art).

Selby et al., American Journal of Epidemiology 125(6):979-88 (1987) argue environmental factors as important to diabetes as they correlate to whether monozygotic twins have or not have diabetes. Likewise, Kesaniemi et al., Acta Genet Med Gemellol (Roma) 33(3): 467-73 (1984).

A correlation between monozygotic twins was noted for obesity as opposed to dizygotic twins. Selby et al., Journal of the American Medical Association 265(16):2079-2084 (1991).

A correlation for monozygotic twins for obesity has been noted by Lemieux, International Journal of Obesity 21(10):831-838 (1997); Obesity in Europe 91, Proceedings of the 3rd European Congress on Obesity, edited by Ailhaud et al., Vol. 062 Abs. No. 04070 John Libbey & Company Ltd. London, UK; Pritchard et al., Metabolism 48(9):1120-7 (1999) (not prior art); Narkiewicz et al., Journal of Hypertension 17(1):27-31 (1999); Pritchard et al., Journal of Clinical Endocrinology and Metabolism 83(9):3277-84 (1989); Hong et al., Arterioscler. Thromb. Vasc. Biol. 17(11):2776-82 (1997); and Oppert et al., Metabolism 44(1):96-105 (1995).

Monozygotic twins who are phenotypically discordant for schizophrenia have been examined by two-dimensional gel electrophoresis, Vander Putten et al., Biol. Psychiatry 40(6):437-442 (1996). The authors report that one protein was significantly elevated between an affected twin and its control twin and that the same protein was significantly elevated when unrelated schizophrenic patients were compared to unrelated normal control individuals.

Carmelli, Heart, Lung, and Blood Institute Award Type-Noncompeting Continuation (Type 5) Fiscal Year-1998 to SRI International, examined monozygotic twins over 23 years of follow-up examining obesity, essential hypertension and non-insulin-dependent diabetes mellitus (NIDDM) for discordant presence.

When comparing monozygotic twins, genetics did not appear to have any substantial effect on obesity and neither appears to account for the variation in hormone values in twin pairs. Meikle et al., Metabolism 37(6):514-7 (1988). Serum lipids, lipoproteins, and lipid metabolizing enzymes in identical twins discordant for obesity were compared.

Monozygotic twins discordant for obesity have been examined regarding certain serum lipoproteins, Ronnemaa et al., Journal of Clinical Endocrinology and Metabolism 83(8):2792-9 (1998), Hayakawa et al., Atherosclerosis 66(1-2):1-9 (1987) and for plasma leptin concentrations, Ronnemaa et al., Annals of Internal Medicine 126(l):26-31 (1997) and Ronnemaa et al., Journal of Clinical Endocrinology and Metabolism 85(8):2728-32 (2000) (not prior art).

### SUMMARY OF THE INVENTION

The object of the instant invention is to discover and to use protein markers for a disease state and the markers per se.

It is a further object of the instant invention to determine protein markers that result from the disease state and which do not appear because of normal genetic variation.

It is another object of the instant invention to provide diagnostic markers for obesity, diabetes, osteoporosis osteoarthritis and hypertension and to diagnose and to stratify these and other diseases by measuring the amount of one or more markers in a biological sample.

It is still a further object of the instant invention to determine the degree of severity of a disease state, its prognosis, the preferred choice of therapy and/or efficiency of therapy by measuring the relative amounts of each of the disease markers and ratios between each and/or other conventional measures of the disease state.

It is yet another object of the instant invention to provide suitable targets for drug discovery of compounds that are agonists or antagonists of a protein and to screen candidate compounds with such targets.

It is another object of the instant invention to compare the relative efficacy of candidate pharmaceuticals and diagnostics by comparing the relative effect on one or more disease marker between candidates or between a candidate and an established pharmaceutical or diagnostic.

It is still another object of the instant invention to determine coregulating proteins that may be used to determine at least parts of a metabolic pathway.

It is another further object of the instant invention to find methods for regulating a first protein by affecting a second protein.

It is yet another further object of the instant invention to determine efficacy of a treatment for a disease by measuring the protein markers during or after treatment and comparing to a positive control, a negative control or the individual before treatment.

It is a still another object of the instant invention to determine sets of presumably related proteins which when taken together constitute a protein marker.

Other aspects of the invention include the protein markers themselves, proteomic displays containing abnormal abundances of the protein markers, and the many uses thereof for research and monitoring patients. Also, combinations of plural proteins constituting a combination marker and submarkers co-fluctuating with other markers may be used as other protein markers.

The instant invention accomplishes those goals by determining which proteins are present in abnormal abundances in biological samples and optionally deducing the mechanism of action from the perturbed metabolic pathway. Initially, all readily detectable proteins are measured; but after the markers are determined, an assay for the markers alone is sufficient. In addition, monitoring of either patients on the drug, laboratory animals in drug discovery or pre-clinical and clinical testing protocols may utilize such an assay. Sets of perturbed protein markers provide a proteomic pattern or “signature” for better determination also indicating aspects of and the status of the diseased state.

The instant invention determined non-genetic disease protein markers by searching for proteins present in abnormal abundances between monozygotic twins where the twins are discordant for the disease state. Initially, all readily detectable proteins are measured in a biological sample to determine which are disease markers; but after the markers are determined, an assay for the markers alone is sufficient for diagnosis. In addition, monitoring of either patients on the drug or laboratory animals in drug discovery or pre-clinical testing protocols for efficacy may utilize such an assay.

### BRIEF DESCRIPTION OF THE DRAWINGS

FIG. 1 is an image of a two dimensional electrophesis gel of human serum master pattern of HUSERFRAC3A-1 with hits highlighted with the MSN spot number.

FIG. 2 is an image of a two dimensional electrophesis gel of human serum master pattern of HUSERFRAC3A-1 populations with groups of hits highlighted with the MSN spot numbers for the individuals and the groups given.

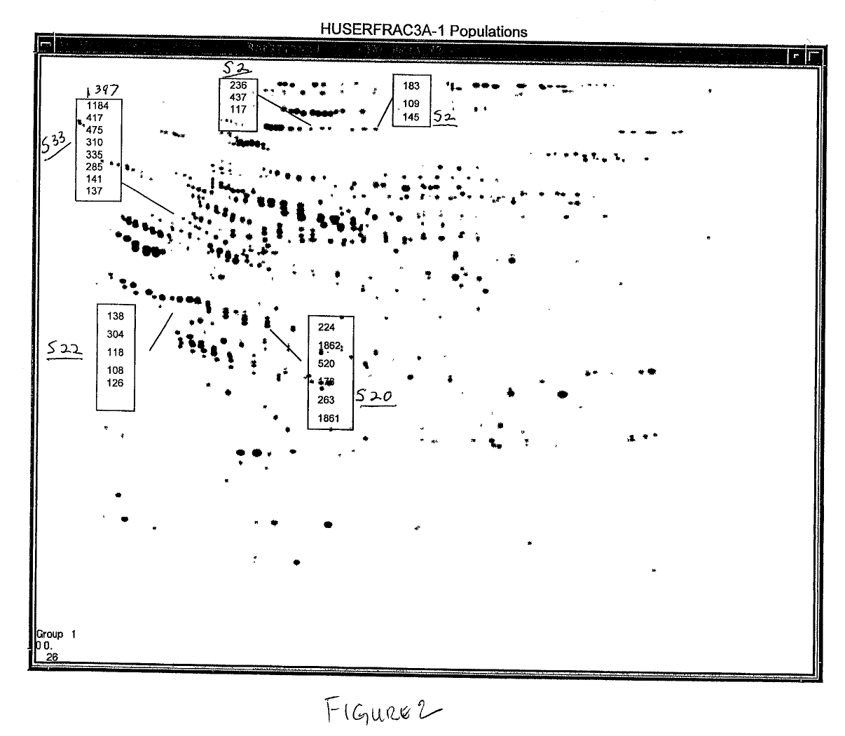

FIG. 3 is an image of a two dimensional electrophesis gel of human serum master pattern of HUSERFRAC3A-2 with hits highlighted with the MSN spot number.

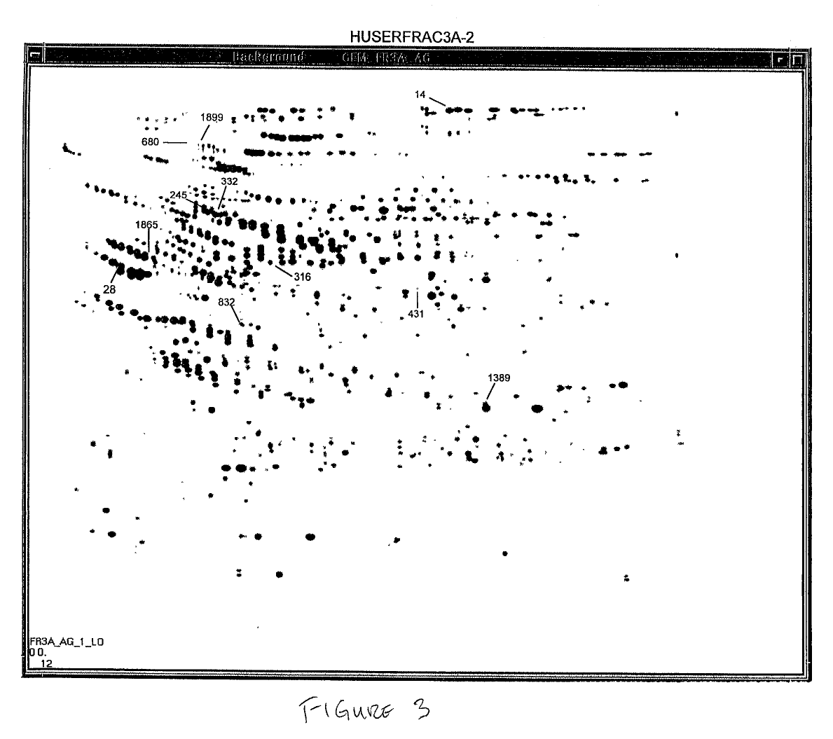

FIG. 4 is an image of a two dimensional electrophesis gel of human serum master pattern of HUSERFRAC3A-2 populations with groups of hits highlighted with the MSN spot numbers for the individuals and the groups given.

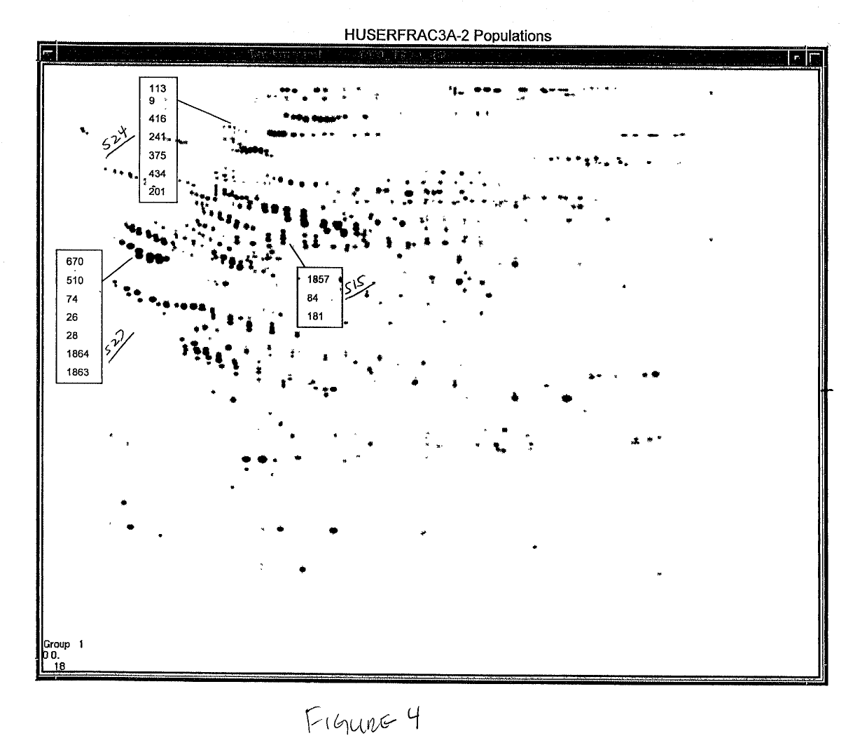

FIG. 5 is an image of a two dimensional electrophesis gel of human serum master pattern of HUSERFRAC5-1 with hits highlighted with the MSN spot number.

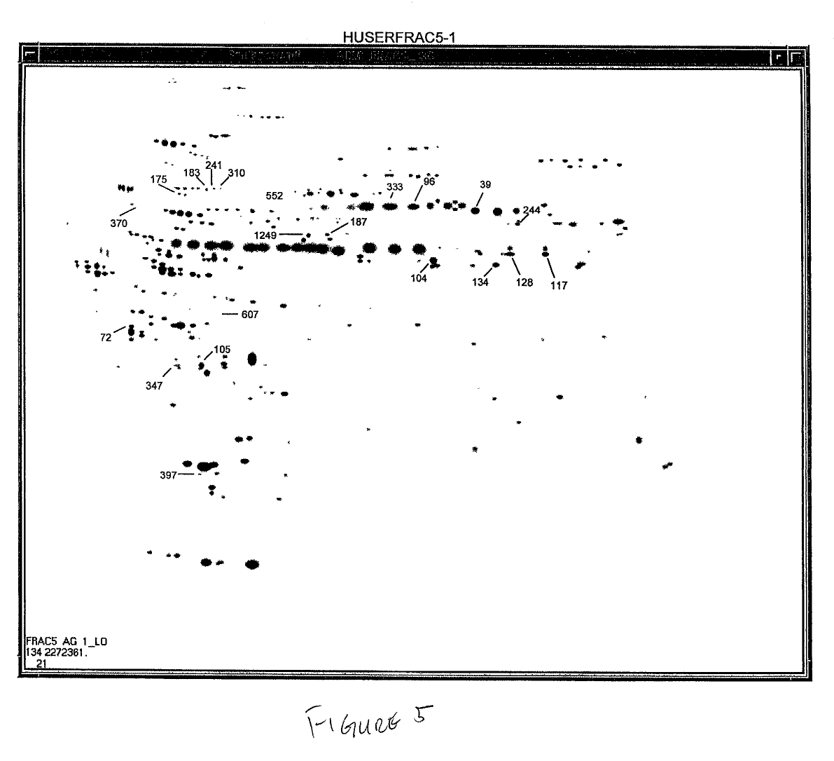

FIG. 6 is an image of a two dimensional electrophesis gel of human serum master pattern of HUSERFRAC5-1 populations with groups of hits highlighted with the MSN spot numbers for the individuals and the groups given.

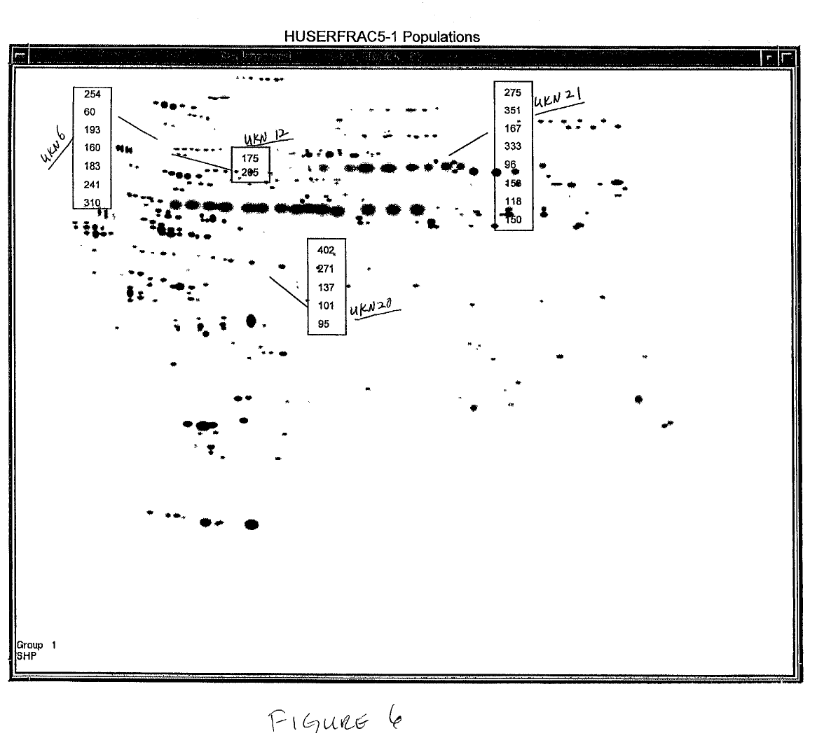

FIG. 7 is an image of a two dimensional electrophesis gel of human serum master pattern of HUSERFRAC5-2 with hits highlighted with the MSN spot number.

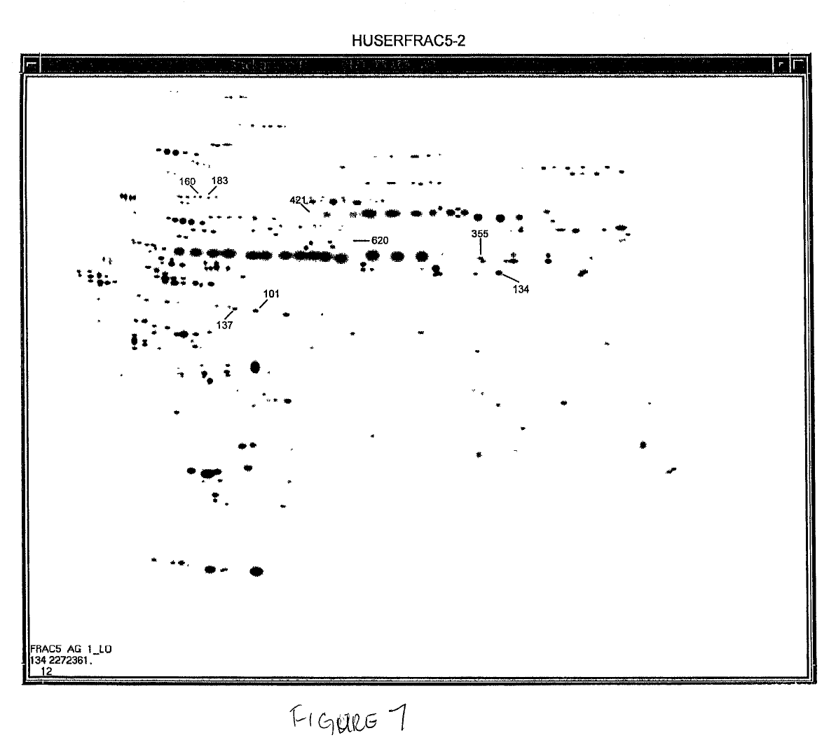

FIG. 8 is an image of a two dimensional electrophesis gel of human serum master pattern of HUSERFRAC5-2 populations with a group of hits highlighted with the MSN spot numbers for the individuals and the groups given.

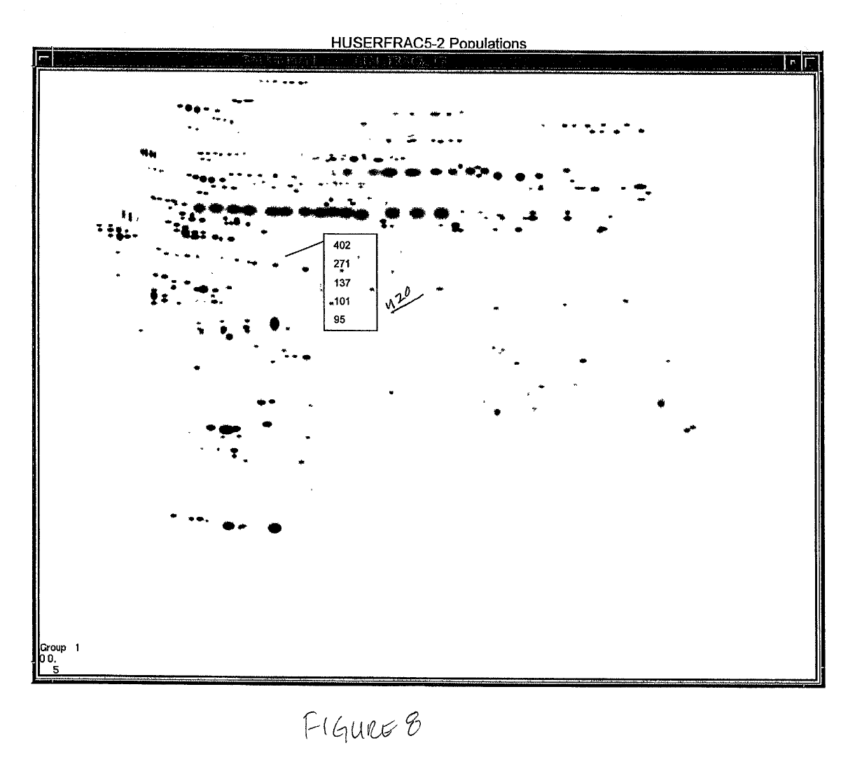

FIG. 9 is an image of a two dimensional electrophesis gel of human serum master pattern of HUSERFRAC6-2 with hits highlighted with the MSN spot number.

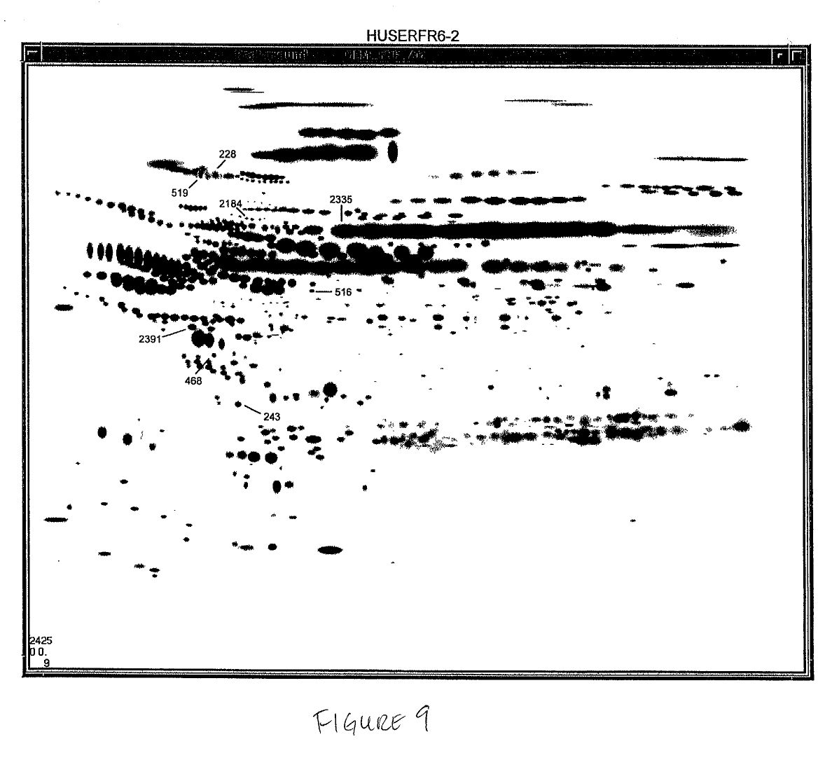

FIG. 10 is an image of a two dimensional electrophesis gel of human serum master pattern of HUSERFRAC5 Alpha group with the ALPHA 1 AT group of hits highlighted with the MSN spot number for the individuals and the group given.

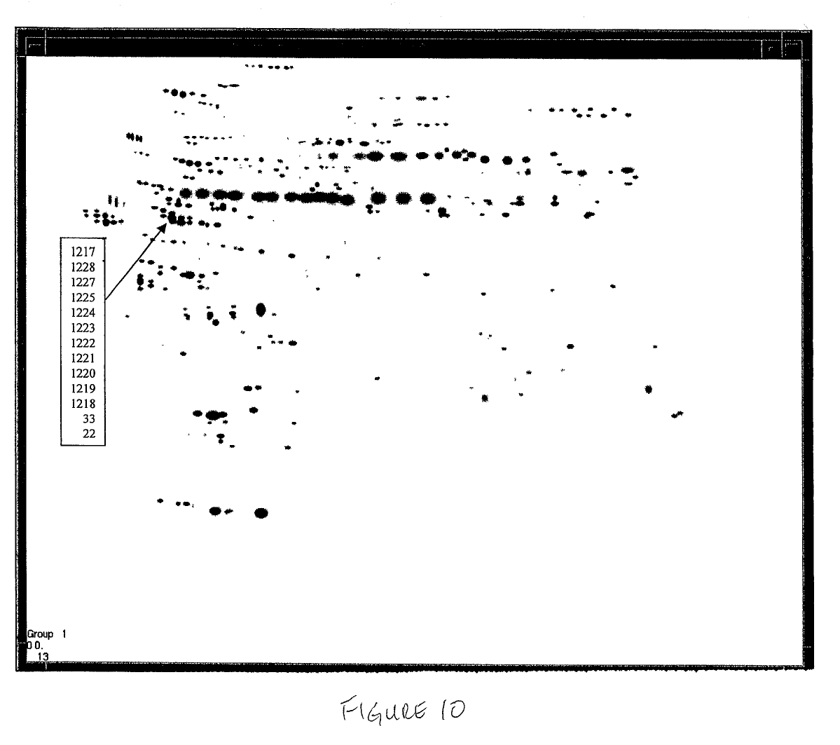

### DESCRIPTION OF THE PREFERRED EMBODIMENTS

The term “diabetes” in the instant application refers to Diabetes Mellitus, Type II or non-insulin-dependent diabetes (NIDDM) or insulin resistance.

The term “isolated”, when referring to a protein, means a chemical composition that is essentially free of other cellular components, particularly most other proteins. The term “purified” refers to a state where the relative concentration of a protein is significantly higher than a composition where the protein is not purified. Purity and homogeneity are typically determined using analytical techniques such as polyacrylamide gel electrophoresis or high performance liquid chromatography. Generally, a purified or isolated protein will comprise more than 80% of all macromolecular species present in the preparation. Preferably, the protein is purified to greater than 90% of all macromolecular species present. More preferably, the protein is purified to greater than 95% and most preferably, the protein is purified to essential homogeneity, or wherein other macromolecular species are not significantly detected by conventional techniques.

The term “protein” is intended to also encompass derivatized molecules such as glycoproteins and lipoproteins as well as lower molecular weight polypeptides.

A “protein marker” is a detectable “protein” which has its concentration, abundance, derivatization status, activity or other level altered in a statistically significant way when a host producing the protein marker has a state that varies from the most prevalent state in a population. Thus the marker, can be, for example, a polymorphism not frequent in a population, a mutation and so on. Some protein markers may be disease-specific.

A “level” refers to abundance, derivatization status, protein variant presence, concentration, chemical activity or biological activity, which is detectable. An “altered level” refers to a change in the “level” when compared to a different sample. The “level” may be an actual measured amount of a protein but is generally a relative “level” of a protein compared to the “level” of other proteins or standards, preferably several hundred from the same experiment.

“Small molecules” are low molecular weight, preferably organic molecules that are recognizable by receptors. Typically, small molecules are specific binding components for proteins.

The terms “binding component”, “ligand” or “receptor” may be any of a large number of different molecules, and the terms are used interchangeably sometimes.

The term “ligands” refers to chemical components in a sample that will specifically bind to receptors. A ligand is typically a protein or peptide but may include small molecules, particularly those acting as a hapten. For example, when detecting proteins in a sample by immunoassay, the proteins are the ligands.

The term “receptors” refers to chemical components in a reagent, which have an affinity for and are capable of binding to ligands. A receptor is typically a protein or peptide but may include small molecules. For example, an antibody molecule acts as a receptor.

The term “bind” includes any physical attachment or close association, which may be permanent or temporary. Generally, an interaction of hydrogen bonding, hydrophobic forces, van der Waals forces etc., facilitates physical attachment between the ligand molecule of interest and the receptor. The “binding” interaction may be brief as in the situation where binding causes a chemical reaction to occur. This is typical when the binding component is an enzyme and the analyte is a substrate for the enzyme. Reactions resulting from contact between the binding component and the analyte are within the definition of binding for the purposes of the instant invention. Binding is preferably specific. The binding may be reversible, particularly under different conditions.

The term “bound to” or “associated with” refers to a tight coupling of two components. The nature of the binding may be chemical coupling through a linker moiety, physical binding or packaging such as in a macromolecular complex. Likewise, all of the components of a cell are “associated with” or “bound to” the cell.

“Labels” include a large number of directly or indirectly detectable substances bound to another compound and are known per se in the immunoassay and hybridization assay fields. Examples include radioactive, fluorescent, enzyme, chemiluminescent, hapten, spin labels, a solid phase, particles etc. Labels include indirect labels, which are detectable in the presence of another added reagent, such as a receptor bound to a biotin label and added avidin or streptavidin, labeled or subsequently labeled with labeled biotin simultaneously or later.

In situations where a chemical label is not used in an assay, alternative methods may be used such as agglutination or precipitation of the ligand/receptor complex, detecting molecular weight changes between complexed and uncomplexed ligands and receptors, optical changes to a surface (e.g., in the Biacore® device) and other changes in properties between bound and unbound ligands or receptors.

An “array” or “microarray” (depending on size) is generally a solid phase containing a plurality of different ligands or receptors immobilized thereto at predetermined locations. By contacting ligands under binding conditions to the microarray, one can determine ligand or receptor identity or at least part of the ligands' structure based on its location on the microarray. While not a single solid phase, a series of many different solid phases (or other labeling structure) each with a unique receptor immobilized thereon is considered a microarray. Each solid phase has unique detectable differences allowing one to determine the ligand or receptor immobilized thereon. An array may contain different receptors in physically separate locations even when they are not bound to a solid phase, for example a multi-welled plate.

The term “disease-related marker or portions thereof” as used herein refers to particular compounds or complexes which are found in abnormal abundances in a disease.

The term “disease state” refers to the disease condition or extent of the condition of an individual. It includes the prognostic situation and other details of the individual's disease, as well which can be used for a variety of indications.

The term “biological sample” includes tissues, fluids, solids (preferably suspendable), extracts and fractions that contain proteins. These protein samples are from cells or fluids originating from an organism. The biological sample may be taken directly from the organism or tissue or indirectly from the organism such as from body fluids such as blood, plasma, serum or urine. In the instant invention, the host is generally an animal, preferably a human, when the diseases are obesity, osteoporosis, diabetes, osteoarthritis or hypertension, and may be any type of organism when discussing disease in general.

The term “proteome” is a large number of proteins expressed in a biological sample, representing the total, relevant portion or preferably all detectable proteins by a particular technique or combination of techniques. “Proteome analysis” is generally the simultaneous measurement of at least 100 proteins, generally at least a few hundred proteins, preferably over 1000 and most preferably plural thousands of detectable proteins from a sample when separated by various techniques. In the instant invention, the proteome analysis involves two-dimensional gel electrophoresis. While this is the generally accepted technique for analyzing proteomes, other techniques are acceptable and may be used for the instant invention if they generate large numbers of quantitatively detectable proteins. Another example is discussed in PCT Patent Application Ser. No. US00/31516.

The term “target” refers to any protein perturbed by a disease, developmental stage or after drug treatment. Frequently, a target refers to a drug development target that is capable of binding, or being altered by, an agent. Such drug development targets are suitable for screening candidate compounds either using direct binding assays or by observing a perturbed level, thereby indicating the candidate compound is appropriate for the next level of drug screening.

The terms “host”, “subject”, “individual”, and “sample of interest” include normal or abnormal organisms, and various tissues, cells and fractions (including subcellular fractions) of each of these.

Monozygotic twins that are discordant for a disease trait represent a good system for studying diseases as the cause cannot be related to any genetic process. Furthermore, monozygotic twins are so identical that almost any difference in their proteins is important. In the instant invention, biological samples are taken from each twin and the quantity and quality of every protein in the biological sample's proteome is compared. When the twins differ dramatically with respect to a particular disease state, the perturbations in the proteome are likely to be caused by the disease state or at least such perturbations represent identifiable markers associated with the disease state.

In the preferred embodiment, the biological samples from many discordant identical twins are subjected to proteometric analysis whereby the quantity of every protein in a twin's sample is compared to its respective partner (if any) in the respective twin sample. The data is analyzed statistically by conventional methods for determining a correlation between each perturbed protein and disease state. The results, given in the tables below are lists of significant markers for each respective disease state.

In many biological samples, a few common proteins constitute a large percentage by weight or mole of all proteins present by weight or mole. These common proteins are typically the least interesting from a disease standpoint and the most interfering. Such common proteins generate large spots and smears on a two-dimensional electrophoresis gel thereby interfering with the measurement of other proteins. To complicate matters, there are limits to how much protein can be run on a two-dimensional electrophoresis gel before the gel becomes fragile and physically breaks into many pieces. Since some of the proteins in the biological samples occur in low concentrations, they may be present below the detectable limit of the system when the common or abundant proteins are present.

To reduce this interference, the common uninteresting proteins are first removed from the protein sample before it is loaded into the electrophoresis separation system. This enhances sensitivity as the protein sample being loaded is depleted of unwanted proteins allowing a higher amount of low abundance proteins to be loaded into the system. That produces a relatively higher amount of such low abundance proteins enhancing their detection.

In the instant invention, this was done by immunosubtraction and/or sample fractionation but a variety of other fractionation methods may be used. By reacting the naturally common proteins to an immobilized specific binding agent such as an antibody, they are effectively and selectively removed from the sample. Furthermore, fractionating the sample mixture into various fractions based on various physical properties, such as the presence or absence of glycosylation, still larger amounts of low abundance proteins may be loaded into the electrophoresis system. Any of a long list of conventional protein separation and purification techniques, well known per se, may be used.

In the preferred embodiment, commercially available solid phase beads (Poros®) having Protein G or Protein A bound thereto and mixtures of them are first contacted with an antibody recognizing one of the proteins. As protein A and protein G bind to the constant region of the antibody molecule, it does not interfere with antibody-antigen binding, thereby maintaining optimal orientation for their affinity towards the serum proteins.

The antibodies are then cross-linked to the Protein G or A after binding by the dimethylpimelimidate method to form stable amide linkages. Schneider et al., Journal of Biological Chemistry 257:10766-10769 (1982). Because interaction between amine groups was minimized due to previous binding of antibody to Protein G or A, the affinity binding sites were minimally affected.

In the instant invention, a correlation having a probability value of p<0.01 was accepted as indicating statistical significance. While p values of less than 0.01 may be considered statistically acceptable to some, markers of greater statistical significance may be more desirable for diagnostic purposes, prognostic purposes, indicative of which therapy to use and for monitoring the effects of therapy to ameliorate the disease state.

Since all p value cut-offs represent a somewhat arbitrary threshold, it is possible and likely to miss significant protein markers using one embodiment of the instant invention. However, by looking at related diseases or artificial disease models, which may be related by mechanism of action, one can find proteins with altered abundance with respect to the controls. Even though not statistically significant alone, if such a protein were found to be altered in biological samples from, for example, an animal model, the result can be considered statistically significant. When determining what is to be considered a protein marker, a protein may constitute a disease marker even when not statistically significant in a single experiment with one causative agent alone.

Total protein markers identified by direct correlation or analysis of variance (ANOVA) are listed below. Proteins were identified by molecular weight, pI, mass of peptides, sequence of peptides and so on. Comparisons were made to various databases, such as the NCBI non-redundant gene sequence database and the SwissProt database.

The protein markers, which are perturbed by various disease states, are as follows. When different variants of the proteins are present and used as markers, references to the different master spot number (MSN) numbers are given.

TABLE 1
Non-Genetic Markers for Obesity

| MSN Number/Master | Protein | Accession |
|---|---|---|
| 19/HUSERFR3A | chain A, cleaved antichymotrypsin | 2982038 |
| 316/HUSERFR3A | | |
| 420/HUSERFR3A | H factor 1 (complement) | 4504375 |
| 501/HUSERFR3A | | |
| 680/HUSERFR3A | | |
| 1139/HUSERFR3A | | |
| 236 (S2)/HUSERFR3A | α₂-macroglobulin precursor | P01023 |
| 138 (S22)/HUSERFR3A | leucine-rich α₂-glycoprotein (Mᵣ = 34346) | P02750 |
| S 24/HUSERFR3A | | |
| 141 (S33)/HUSERFR3A | SNC73 protein | 3201900 |
| 519/HUSERFR6 | | |
| 39/HUSERFRAC5 | | |
| 72/HUSERFRAC5 | complement component 3 precursor | 4557385 |
| 96/HUSERFRAC5 | immunoglobuim M heavy chain (Homo | 4467842 |
| | sapiens) | |
| 101/HUSERFRAC5 | pigment epithelial-differentiating factor | 284355 |
| | precursor (human) | |
| 104/HUSERFRAC5 | apolipoprotein H (human) | P02749 |
| 105/HUSERFRAC5 | | |
| 117/HUSERFRAC5 | complement factor B precursor | P00751 |
| 128/HUSERFRAC5 | complement factor B precursor | P00751 |
| 134/HUSERFRAC5 | apolipoprotein H (human) | P02749 |
| 137/HUSERFRAC5 | pigment epithelial-differentiating factor | 284355 |
| | precursor (human) | |
| 175/HUSERFRAC5 | | |
| 241/HUSERFRAC5 | complement component C4B (Homo sapiens) | 187771 |
| 333/HUSERFRAC5 | Ig heavy chain (human) | 106378 |
| 355/HUSERFRAC5 | | |
| 370/HUSERERAC5 | complement component C4B (Homo sapiens) | 187771 |
| 397/HUSERFRAC5 | | |
| 421/HUSERFRAC5 | | |
| 552/HUSERFRAC5 | | |
| 607/HUSERFRAC5 | | |
| 890/HUSERFRAC5 | | |
| ALPHA 1/HUSERFRAC5 | | |
| UK4 12/HUSERFRAC5 | | |
| UKN 20/HUSERFRAC5 | | |

TABLE 2
Genetic Markers for Osteoporosis

| MSN Number/Master | Protein | Accession |
|---|---|---|
| 36/HUSERFR3A | | |
| 128/HUSERFR3A | hemopexin precursor (β_{1B}-glycoprotein) (human) | P02790 |
| 152/HUSERFR3A | hemopexin precursor (β_{1B}-glycoprotein) | P02790 |
| | (Mᵣ = 51676) | |
| 245/HUSERFR3A | pk 120 precursor | 1402590 |
| 431/HUSERFR3A | | |
| 228/HUSERFR6 | | |
| 243/HUSERFR6 | 60 kd heat shock protein, mitochondrial (human) | P 10809 |
| 516/HUSERFR6 | antithrombin Ill variant | 576554 |
| 160/HUSERFRAC5 | complement component C4B (Homo sapiens) | 187771 |
| 183/HUSERFRAC5 | complement component C4B (Homo sapiens) | 187771 |
| 187/HUSERFRAC5 | heparin cofactor II precursor P05546 | |
| 244/HUSERFRAC5 | | |
| 310/HUSERFRAC5 | complement component C4B (Homo sapiens) | 187771 |
| 607/HUSERFRAC5 | | |
| 620/HUSERERACS | | |
| 856/HUSERFRAC5 | | |
| 1042/HUSERFRAC5 | | |
| 1249/HUSERERAC5 | heparin cofactor II precursor | P05546 |
| UKN 12/HUSERFRAC5 | | |
| UKN 6/HUSERFRAC5 | | |

TABLE 3
Non-Genetic Markers for Diabetes

| MSN Number/Master | Protein | Accession |
|---|---|---|
| 14/HUSERFR3A | | |
| 28/HUSERFR3A | α₂-HS glycoprotein | 4502005 |
| 332/HUSERFR3A | inter-α(globulin) inhibitor H4 | 4504785 |
| 832/HUSERFR3A | | |
| 1389/HUSERFR3A | | |
| 1865/HUSERFR3A | Kininogen | 386852 |
| 181 (S15)/HUSERFR3A | SNC 73 Protein | 3201900 |
| 26/44 (S27)/HUSERFR3A | α₂-HS glycoprotein | 4502005 |
| 101/HUSERFRAC5 | pigment epithelial-differentiating | 284355 |
| | factor precursor (human) | |
| precursor (human) | | |

TABLE 4
Non-Genetic Markers for Osteoarthritis
MSN Number/Master
1992/HUSERFR6

TABLE 5
Non-Genetic Markers for Hypertension
MSN Number/Master/Protein

| MSN Number/Master | Protein | Accession |
|---|---|---|
| 520/HUSERFR3A | haptoglobin-1 precursor | P00737 |
| | (Mᵣ = 38452) | |
| 1862/HUSERFR3A | haptoglobin-1 precursor | P00737 |
| | (Mᵣ = 38452) (human) | |
| 224/263 (S20)/USERFR3A | haptoglobin-1 precursor | P00737/ |
| | (Mᵣ 38452) | 2118089 |
| 468/HUSERFR6 | | |
| 2184/HUSERFR6 | | |
| 2335/HUSERFR6 | immunoglobulin M heavy chain | 4467842 |
| | (Homo sapiens) | |
| 2391/HUSERFR6 | | |
| 347/HUSERFRAC5 | | |
| 800/HUSERFRAC5 | | |

TABLE 6
Identification of the Protein Spots

| MASTER | MSN | stain | trait | type | pi | mw |
|---|---|---|---|---|---|---|
| HUSERFR3A | 14 | AG | INSRES_z | correlation | 6.22 | 139364 |
| HUSERFR3A | 19 | CB | TFM_z | anova | 4.12 | 70122 |
| HUSERFR3A | 28 | AG | INSRES_z | anova | 4.2 | 61684 |
| HUSERFR3A | 36 | AG | SBMD_z | correlation | 5.12 | 82460 |
| HUSERFR3A | 128 | CB | TOTBMD_z | anova | 5.57 | 74350 |
| HUSERFR3A | 152 | CB | TOTBMD_z | anova | 5.98 | 73753 |
| HUSERFR3A | 245 | AG | SBMD_z | correlation | 5.07 | 82737 |
| HUSERFR3A | 316 | AG | PCTFAT | correlation | 5.51 | 63087 |
| HUSERFR3A | 332 | AG | INSRES_z | anova | 5.13 | 81527 |
| HUSERFR3A | 420 | CB | TFM_z | correlation | 5.1 | 109205 |
| HUSERFR3A | 431 | AG | TOTBMD_z | correlation | 6.14 | 55277 |
| HUSERFR3A | 501 | CB | TFM_z | anova | 5.64 | 30450 |
| HUSERFR3A | 520 | CB | RCAI | correlation | 5.22 | 42485 |
| HUSERFR3A | 680 | AG | TFM_z | correlation | 4.99 | 116640 |
| HUSERFR3A | 832 | AG | INSRES_z | correlation | 5.33 | 44792 |
| HUSERFR3A | 1139 | AG | TFM_z | correlation | 4.95 | 117289 |
| HUSERFR3A | 1389 | AG | INSRES_z | correlation | 6.31 | 29942 |
| HUSERFR3A | 1862 | CB | RCAI | correlation | 5.09 | 43239 |
| HUSERFR3A | 1865 | AG | INSRES_z | correlation | 4.48 | 64489 |
| HUSERFR3A | S15 | AG | INSRES_z | anova_pop_position | | |
| HUSERFR3A | S2 | CB | TFM_z | cor_pop_position | | |
| HUSERFR3A | S20 | CB | RCAI | cor_pop_average | | |
| HUSERFR3A | S22 | CB | TFM_z | cor_pop_position | | |
| HUSERFR3A | S24 | AG | TFM_z | cor_pop_position | | |
| HUSERFR3A | S27 | AG | INSRES_z | anova_pop_average | | |
| HUSERFR3A | S33 | CB | TFM_z | anova_pop_average | | |
| HUSERFR6 | 228 | AG | TOTBMD_z | correlation | 5 | 113862 |
| HUSERFR6 | 243 | AG | TOTBMD_z | correlation | | 31818 |
| HUSERFR6 | 468 | AG | RCAI | correlation | | 39037 |
| HUSERFR6 | 516 | AG | TOTBMD_z | correlation | 5.55 | 54703 |
| HUSERFR6 | 519 | AG | PCTFAT | correlation | 4.89 | 113581 |
| HUSERFR6 | 1992 | AG | OVE_z | correlation | 4.99 | 31420 |
| HUSERFR6 | 2184 | AG | CCAI_z | anova | 5.21 | 85763 |
| HUSERFR6 | 2335 | AG | CCAI_z | anova | 5.68 | 79223 |
| HUSERFR6 | 2391 | AG | RCAI | correlation | 4.82 | 44734 |
| HUSERFRAC5 | 39 | CB | TFM_z | correlation | 6.45 | 78358 |
| HUSERFRAC5 | 72 | CB | TFM_z | anova | 4.94 | 42730 |
| HUSERFRAC5 | 96 | CB | TFM_z | correlation | 6.19 | 79944 |
| HUSERFRAC5 | 101 | AG | INSRES_z | anova | 5.59 | 47373 |
| HUSERFRAC5 | 101 | AG | TFM_z | correlation | 5.59 | 47373 |
| HUSERFRAC5 | 104 | CB | TFM_z | correlation | 6.27 | 58175 |
| HUSERFRAC5 | 105 | CB | TFM_z | correlation | 5.35 | 37624 |
| HUSERFRAC5 | 117 | CB | TFM_z | correlation | 6.82 | 60637 |
| HUSERFRAC5 | 117 | CB | PCTFAT | correlation | 6.82 | 60637 |
| HUSERFRAC5 | 128 | CB | TFM_z | correlation | 6.62 | 60420 |
| HUSERFRAC5 | 134 | AG | TFM_z | correlation | 6.55 | 57014 |
| HUSERFRAC5 | 134 | CB | TFM_z | correlation | 6.55 | 57014 |
| HUSERFRAC5 | 137 | AG | TFM_z | correlation | 5.5 | 47784 |
| HUSERFRAC5 | 160 | AG | SBMD_z | correlation | 5.34 | 88883 |
| HUSERFRAC5 | 175 | CB | TFM_z | correlation | 5.24 | 85833 |
| HUSERFRAC5 | 183 | AG | SBMD_z | correlation | 5.38 | 88728 |
| HUSERFRAC5 | 183 | CB | SBMD_z | correlation | 5.38 | 88728 |
| HUSERFRAC5 | 187 | CB | SBMD a | correlation | 5.87 | 67324 |
| HUSERFRAC5 | 241 | CB | TFM_z | anova | 5.42 | 88879 |
| HUSERFRAC5 | 244 | CB | TOTBMD_z | anova | 6.67 | 72180 |
| HUSERFRAC5 | 310 | CB | SBMD a | correlation | 5.45 | 89277 |
| HUSERFRAC5 | 333 | CB | TFM_z | correlation | 6.1 | 79895 |
| HUSERFRAC5 | 347 | CB | RCAI | correlation | 5.22 | 37586 |
| HUSERFRAC5 | 355 | AG | TFM_z | correlation | 6.46 | 61569 |
| HUSERFRAC5 | 370 | CB | TFM_z | correlation | 5 | 79599 |
| HUSERFRAC5 | 397 | CB | PCTFAT | correlation | 5.35 | 29723 |
| HUSERFRAC5 | 421 | AG | TFM_z | correlation | 5.81 | 80243 |
| HUSERFRAC5 | 552 | CB | TFM_z | anova | 5.74 | 79735 |
| HUSERFRAC5 | 607 | CB | TFM_z | correlation | 5.42 | 44992 |
| HUSERFRAC5 | 607 | CB | SBMD_z | correlation | 5.42 | 44992 |
| HUSERFRAC5 | 620 | AG | SBMD_z | correlation | 5.94 | 67780 |
| HUSERFRAC5 | 800 | AG | RCAI | correlation | 5.23 | 28230 |
| HUSERFRAC5 | 856 | AG | SBMD_z | correlation | 6.03 | 71694 |
| HUSERFRAC5 | 890 | AG | TFM_z | anova | 6.89 | 32227 |
| HUSERFRAC5 | 1042 | AG | SBMD_z | correlation | 6.24 | 70636 |
| HUSERFRAC5 | 1249 | CB | SBMD_z | correlation | 5.81 | 66964 |
| HUSERFRAC5 | ALPHA1 | AT | TFM_z | anova_pop_average | | |
| | | CB | | | | |
| HUSERFRAC5 | UNK12 | CB | SBMD_z | anova_pop_average | | |
| HUSERFRAC5 | UNK12 | CB | TFM_z | cor_pop_average | | |
| HUSERFRAC5 | UNK20 | AG | TFM_z | cor_pop_average | | |
| HUSERFRAC5 | UNK20 | CB | TFM_z | cor_pop_average | | |
| HUSERFRAC5 | UNK21 | CB | TFM_z | cor_pop_average | | |
| HUSERFRAC5 | UNK6 | CB | SBMD_z | cor_pop_average | | |
| HUSERFRAC5 | UNK6 | CB | SBMD_z | anova_pop_position | | |

TABLE 7
LCQ MS/MS spectra

| Fraction | 5 | 5 | 6 |
|---|---|---|---|
| MSN | 105 | 39 | 243 |
| m/z selected | 730 | 729 | 511.3* |
| for MS/MS | 408.5 | 627.3 | 9757* |
| | 485.5 | 561.6 | 525 |
| | 764 | | 885.3 |
| | 757.4 | | 383 |
| | 562.9 | | |
| | 403.4 | | |
| | 482.4 | | |

*Doubly-charged masses seen in corresponding MALDI spectra as +1 ions

The pI and MW are predicted from the warped and impressed locations on the two dimensional gel. The precision is generally very good but may vary as much as approximately ±0.5 pI units and ±10% in molecular weight.

The ANOVA analysis sought consistent increase or decrease in protein abundance for the twin with the higher expression of the disease trait. This was a two-way ANOVA analysis based on the following simultaneous criteria: probability is <0.01, binary twin (lower twin value=0, higher twin value=1) is <0.01 and N (number of discordant twin pairs)>8 subjects. The overall correlation criteria is p<0.01 for Pearson correlation between trait and protein spot volume, p<0.01 for Spearman correlation between trait and protein spot volume, p<0.01 for Spearman correlation between trait and protein spot volume (discordant pair subjects), p<0.01 for Pearson correlation between twins, and p<0.01 for Spearman correlation between twins. In Table 6, correlation is linkage of a protein with the disease state or trait in one of a pair of twins; cor is correlation; pop position is a zone or area that defines a plurality of related proteins, generally the same protein, wherein the individual spots represent variants of the protein that carry additional moieties and varying charge, thus, glycosylation increases molecular weight but not necessarily pI and phosphoylation may not yield a noticeable change in molecular weight but could alter pI; pop is population; and pop average is an average molecular weight and PI for a population of related proteins that likely are the same protein but with modifications that alter molecular weight and/or pI.

Even though the protein may not be heretofore isolated or characterized, the instant invention effectively isolates and characterizes the proteins. From the MSN number given above, one has a unique isolated protein from a spot on the 2-dimensional electrophoresis gel. The relative molecular weight and relative pI for each spot are determinable by reference to established landmark proteins, which are fully characterized by sequencing, and a theoretical molecular weight and pI calculated. By plotting the theoretical values on a graph and comparing the location of the previously unknown spot, these identifying features are determined. See Anderson et al., Electrophoresis 16:1977-1981 (1995) for more details, the contents of which are specifically incorporated by reference. This provides a reproducible method for isolating the protein markers of the instant invention.

To confirm the landmarks, biological samples from the experimental subjects were mixed with very well characterized biological samples before separation and quantification. By co-separating them, and comparing the results with the very well characterized biological sample proteins, one may confirm identification of a common protein and/or extrapolate pI and molecular weight values for each spot.

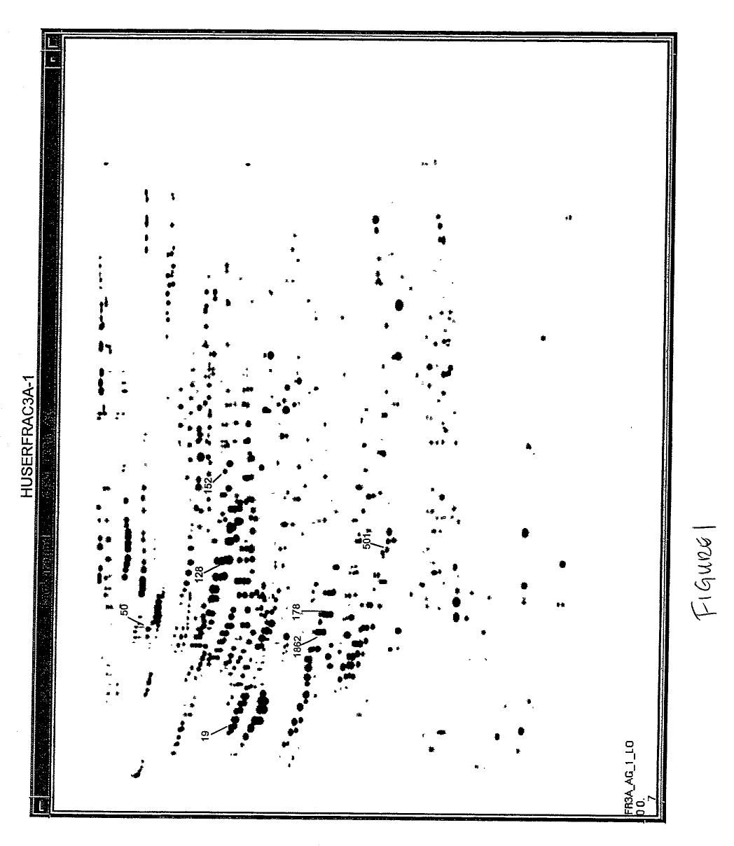

The FIGS. 1 -10 show the placement of each spot relative to other spots in the two-dimensional electrophoresis gel.

While it is very useful to know the quantities of various protein ligands in a sample, in some situations, it may be useful to compare the sample to a standard or to measure differences in concentrations of various ligands from another sample. For example, disease specific makers may be deduced by determining which proteins are in higher or lower concentrations in a sample from a diseased individual as compared to a normal individual. The differential may be determined by using the instant invention to determine the quantities in a normal and a diseased sample. The results from each experiment are compared to generate the differential results.

A particular protein level may be compared to total protein levels in the sample if a concentration control is desired. This will generate a coefficient to compare to standards so that control need not be run side by side every time. Total protein may be determined by measuring total protein being loaded on the gel, but preferably, it is compared to all other spots in the 2DE gel or even total protein in a sample. Alternatively, one may compare a particular protein to a standard protein in the sample (natural internal control) or added to the sample (added internal control).

Proteomic techniques were used to study proteome changes in biological samples from diseased and normal genetically identical twins. The diseases were found to induce a complex pattern or “signature” of alterations in biological sample proteins, some of which are probably related to the disease process and others simply unrelated markers altered by the individual's response to the disease state.

Numerous changes in the proteome of serum from individuals with obesity, osteoporosis, diabetes, osteoarthritis and hypertension were observed. For obesity, total fat mass (TFM) and percent fat (PCTFAT) (fat mass/body weight) were measured. For diabetes, insulin resistance (INSRES), fasting insulin levels and central fat mass were measured. For osteoporosis, bone density, spine bone mass density (SBMD) and total bone mass density (TOTBMD) were measured. For osteoarthritis, joint space narrowing as detected by X-ray or overall score (OVE) (hip joint gap measurement) were measured. For hypertension/arterial distensibility, the following were measured arterial tonometry, pulsewave velocity, central CAI (CCAI) and radial CAI (RCAI), blood pressure measures.

When determining what is to be considered a protein marker, combinations of proteins may constitute a combination marker of disease state or efficacy of treatment. Even when two or more proteins are not sufficiently statistically significant to be considered markers by themselves, when considered in combination, the combination marker may be statistically significant. This is done by determining proteins that are at altered abundances in biological samples from diseased states compared to normal controls. Selecting two proteins that are less than statistically significant markers by themselves, one may combine the values in various ways for two or more of these proteins and determine whether the combination of values is altered in a statistically significant manner. Combination markers result when statistically significant differences between biological samples from diseased individuals and biological samples from control individuals are determined. Suitable data mining reveals a number of combination markers, and the theoretical rationale for some of these combination markers is still being determined.

Testing samples from subjects treated with therapeutics having different mechanisms of action is particularly preferred when searching for new candidate drugs of potentially new mechanisms of action. Markers common with different therapeutics represent a secondary pharmaceutical function. By comparing protein marker effects when using different pharmaceuticals, less than statistically significantly changed proteins may become protein markers of therapeutic benefit.

An index marker is similar to a combination marker except that each protein in the index is itself already statistically significant as a protein marker alone. An index marker is an aggregate of plural significant protein markers which taken together and compared to the same index marker of a different sample. The index marker is then an extremely significant combination. For example, using a combination of markers, each with p<0.001, may yield an index marker of p<0.00001 or lower.

Protein markers found altered by the same general disease but by different causes represent different categories. Producing the same markers are perhaps the best markers for screening new candidate drugs for a given indication because they are not mechanism of action-specific. These are believed to reveal elements common to the mechanisms of action of the different pharmacological classes on a particular disease state. Such a marker is good for screening for drugs having completely unknown modes of action but directed to a similar disease treatment objective.

By using a different method for measuring the proteome, different markers may also be uncovered. Presently two-dimensional electrophoresis is the preferred method for measuring the proteins in a proteome. However other techniques such as a plurality of different chromatography methods may be used. Even within a preferred method for measuring proteins in a proteome, different variations reveal different proteins. For example, different protein solubilizing solutions and different gradients affect which proteins will be observed on a two-dimensional electrophoresis gel. Furthermore, by comparing how one protein changes in abundance with respect to others, still other protein markers may be found.

This method is performed by comparing all proteins that change in abundance in the same or opposite direction as known protein markers. Even if the change in abundance of the proposed protein marker is not changed significantly, the fact that its abundance changes along with established protein markers indicates it may be an acceptable marker.

Another method for finding a marker even when the data is not statistically significant is to determine whether a protein is altered in tandem with known protein markers. Proteins that are not sufficiently altered, to be considered protein markers, are called protein “submarkers” when they have altered levels in tandem or opposite direction and magnitude when consistent among a group of samples. The direction and amount of alteration between the control and disease samples is noted. This is compared across multiple individuals and compared to established protein markers. Tandem moving protein submarkers that are altered both in direction and in amount between individuals and paralleling known protein markers may then be considered to be “protein markers” in their own right. Such may then be assayed for the multitude of purposes as any other marker.

Another method for measuring the proteins in a two-dimensional electrophoresis gel is by determining qualitatively whether a protein is present or absent. For example, a protein found in a biological sample from a control but not in a comparable sample from a disease sample would be of particular interest as it represents that the disease state completely eliminated the protein marker. Likewise, the reverse where a protein is induced only in the diseased state but not present in controls is also of particular interest. A p value is not even calculable in these situations as one is comparing to zero.

Another qualitative or quantitative change in protein marker levels is in the presence of or amount of protein variants and the ratios between them. Some disease states are known to alter glycosylation and any candidate compound being tested may induce a different abundance of protein variants. Likewise, cleavage fragments (or the lack thereof) may be in altered abundance. Still further, enzymes may be in the same concentration but have dramatically different activity due to various agents such as cofactors, metal ions, vitamins etc. In all of these situations, the altered level or change in abundance of a protein or its variant(s) may be used to serve as a suitable marker for disease status. This may be observed as a shift in spot location or new spot formation.

Some diseases actually have different causes and may result in different markers. For example, a headache may be caused by literally dozens of different problems. To best determine which apparently of the same disease have an unrelated mechanism, it is desirable to compare to a composite effect of many drugs and other therapeutic agents, preferably from a large proteomics database. The comparison to the positive control same mechanism of action and the negative control same mechanism of action may be seen as agonist/antagonist effects and correlations between these two control groups provides a further source for protein markers.

A fingerprint of a protein can be obtained by fragmenting the protein, using, for example, an enzyme, and determining the molecular weight of the fragments. That exercise can be performed using mass spectrometry (MS), such as MALDI MS. Thus, a protein can be digested with trypsin and then the sizes of the trypsin fragments determined. An example of such results is presented in Table 7 above. Individual oligopeptides can be sequenced, using known techniques, such as, forms of MS or Edman degradation. The amino acid sequences of the fragments, that is, partial sequences of an intact protein, may be diagnostic for a particular protein. Such comparisons can be made manually or using known programs.

Often a protein may be represented as multiple spots on a gel. Such occurrances can arise because of a variety of phenomenon including post-translational modification, including glycosylation, truncation and phosphorylation, allelism and so on.

Diagnostic uses for the markers are not limited to measuring the proteome for each biological sample. Once one or several critical markers are determined, these proteins alone may be assayed as a way to test for diagnosing the disease state, its prognosis, treatment choices and monitoring response. A number of protein assays are known per se and they vary depending on the protein being measured. Of particular interest are immunoassays as they are fast, inexpensive and relatively simple to perform.

When a protein is diagnostic for a disease, the protein can be analyzed and identified using known techniques. It will be appreciated however, that identification of a protein is not necessary for the protein to be used, for example, in a diagnostic fashion. Thus, a diagnostic protein can be isolated from a 2-D gel by removing a protein spot from the gel. A gel plug containing the protein of interest is crushed in a buffer to enable the protein to diffuse from the gel. The protein is separated and concentrated to yield a purified sample of the protein of interest. The protein then can be used as needed, for example, for making an antibody.

The preparation of antibodies to known isolated proteins is well known per se. In the instant invention, it may be useful to prepare both monospecific antisera, which ideally will be affinity purified before use, and monoclonal antibodies. For diagnostic purposes, monoclonal antibodies are usually preferred to enhance uniformity and specificity. For immunosubtraction procedures, antisera containing polyclonal antibodies is usually preferred as total antigen binding is what is most critical and multiple antibody clones provide enhanced binding. Other specific ligands may be used such as recombinant antibodies, single chain antibodies, antibody display phage, selected members from combinatorial libraries and the like.

Diagnostic reagents and kits of the instant invention are typically used in a “sandwich” format to detect the presence or quantity of proteins in a biological sample. A description of various immunoassay techniques is found in “Basic and Clinical Immunology” (4th ed. 1982 and more recent editions) by D. P. Stites at al., published by Lange Medical Publications of Los Altos, Calif., and in a large number of patents including U.S. Pat. Nos. 3,654,090, 3,850,752 and 4,016,043, the respective contents of which are incorporated herein by reference.

In a preferred embodiment, the kit further includes, a labeled component that is bound to or is bindable to the detection reagents or the protein being assayed or both. Also, in a separate package, an amplifying reagent such as complement, such as guinea pig complement, anti-immunoglobulin antibodies or Staphylococcus aureus Cowan strain protein A that reacts with the antigen or antibodies being detected. In these embodiments, the label specific binding agent is capable of specifically binding the amplifying means when the amplifying means is bound to the protein or antibody.

Important to the labeling and detection systems is the ability to determine quantity of label present to quantify the ligands present in the original sample. Since the signal and its intensity is a measure of the number of molecules bound from the sample and hence of the number of receptors bound, the number of ligand molecules in the original sample may be determined. Optical and electrical signals are readily quantifiable. Radioactive signals may also be quantifiable directly but preferably are determined optically by use of a standard scintillation cocktail.

While the receptors most commonly utilized are antibody molecules, or a portion thereof, one may equally use other specific binding receptors such as hormone receptors, intracellular signal receptors, certain cell surface proteins (also called receptors in the scientific literature), an assortment of enzymes, signal transduction proteins and binding proteins found in biological systems.

Likewise, ligands exemplified as proteins below may also be small organic molecules such as metabolic products in a cell. By simultaneously detecting many or all metabolites in a sample, one can determine the global effects of an effector on the cell. Effectors may be the disease state itself, drugs, toxins, infectious agents, physiological stress, environmental changes etc.

As the number of markers found is large, a simultaneous multiple assaying systems such as a microarray of binding agents for each desired protein marker is preferred. In such a microarray, a specific binding receptor for each protein marker ligand, e.g. an antibody, is immobilized at a different address and contained in a distinct region of the microarray or bound to a distinct particle or label. The protein marker ligand containing sample is then contacted to the microarray and allowed to bind. Binding may then be detected by a number of techniques, known per se, particularly preferred being binding a labeled receptor to one or more components of a ligand/receptor complex and detecting the label.

Microarrays containing multiple receptors are known per se. A test strip with multiple receptors is available commercially. A number of designs for multiple simultaneous binding assays are known per se in the analytic testing field.

The array may utilize antibody or receptor display phage expressing antibody or other receptor as a binding agent or an immobilizing agent for the protein marker ligand. Either the receptor alone or the whole display phage may be used. When used as an immobilizing agent, different cells of the microarray contain a different receptor. When used as a labeled binding agent, the receptor or phage may be labeled (before or after binding to the ligand) by a number of techniques (such as direct fluorescent dyes, e.g. TOTO-1, labeled protein A or G, labeled anti-Ig etc.) and utilized without prior identification of which display phage contains a particular antibody as an initial immobilized capture receptor assay for discrimination.

Other competitive techniques using a microarray of immobilized protein markers and labeled or labelable receptors may also be used.

The techniques described in PCT patent application Ser. No. US00/31516 may be employed to measure a very large number of proteins simultaneously, including any or all of those in a pathway relating to a disease state. Such a technique may be applied to detecting any or all of the protein markers of the instant invention.

For microarrays that are not a unitary solid phase, multiple different beads, each with a different label or having a different combination of labels may be used. For example, a bead having different shades of a chromagen or different proportions of different chromagens or other detectable features can be used. Each bead or set of beads with the same identifying label(s) is to have an immobilized ligand or receptor. Individual sets of beads may be identified in a mixture by spreading on a flat surface and scanning or by moving the beads past a detector. The combination of the labels and the bead label(s) provides identification of the ligand of interest in the sample. The numerical ratio of beads having labels to beads without labels or with different labels provides a quantitative measurement. Just as the sample may be deduced from which addresses contained labels in a traditional microarray, with plural unique beads, the address may be deduced by determining which bead contains the corresponding label(s).

Once the isolated protein on a two-dimensional electrophoresis gel or in other isolated form is obtained, the protein may be identified. If the protein is known, such as those identified in the tables hereinabove. and the gene cloned, one can then produce large quantities of protein by conventional recombinant DNA methods. Likewise, if the protein is not known or is known but the gene not cloned, the amino acid sequence may then be determined by sequencing, mass spectrometry or other methods well known per se. One may deduce the possible nucleotide sequences from the amino acid sequence and use such probes to isolate the gene using well-known techniques known per se to obtain the gene.

Thus, an isolated protein can be sequenced using known techniques. The protein can be fragmented, for example, by proteases, peptidases or other forms of hydrolysis, prior to sequencing. Partial sequences, that is, a sequence of a fragment, generally are sufficient for determining identity with proteins contained in databases. Should there be no matches, accounting for allelic variation, the sequencing can be conducted to completion. The sequence of the protein can be one of a newly uncovered protein or can be one of a known protein yet to be sequenced.

As an alternative to, or preferable in conjunction with, measuring the amount of a protein marker of interest in a biological sample, one may also measure the level of mRNA for the protein marker. This level of mRNA may be measured in absolute levels or relative to all other or specific other mRNA. One may even correlate between protein concentrations and mRNA concentrations if so desired.

If the protein is one that is known, it is possible then to utilize properties of the marker itself for diagnostic and therapeutic purposes. Thus, for example, when the protein is an enzyme, a bioassay based on the activity of the enzyme can be practiced. Hence, a labeled substrate can be monitored for change into a product by the action of the enzyme.

Moreover, the protein may be found to have a causal relationship to the disease. For example, overexpression of a protein may be directly responsible for a disorder. Accordingly, developing ways to reduce the excessive levels of protein can be therapeutic. Ways to achieve that goal include altering the regulation of the gene encoding the protein so that lower levels of protein message are transcribed or translated, using methods known in the art.

For therapeutic purposes, pharmaceutical compositions in the form of small organic molecules, peptides, proteins, antibodies or other specific binding receptors, which may act as agonists or antagonists for the protein markers, may be used. The protein constituting the marker itself may be a functional active ingredient as well. Compositions that regulate expression of the gene encoding the marker, such an antisense molecules may also be used. Each of these classes of pharmaceuticals has been used previously against other drug discovery targets and is thus likely that results will be obtained from the drug discovery targets offered by the instant invention.

Pharmaceutical compositions may be prepared for use in humans or animals via the oral, parenteral, aerosol or rectal route, in the form of wafers, capsules, tablets, gelatin capsules, powders, drinkable solutions, injectable solutions, including delayed-release forms and sustained-release dressings for transdermal administration of the active principle, nasal sprays, or topical formulations (cream, emulsion etc.), comprising a compound interacting with a marker of the instant invention and at least one pharmaceutically acceptable carrier. The pharmaceutical compositions according to the instant invention are advantageously dosed to deliver the active principle in a single unit dose.

For oral administration, the effective unit doses are between 0.1 μg and 500 mg. For intravenous administration, the effective unit doses are between 0.1 μg and 100 mg.

According to the instant invention, the pharmaceuticals are preferably administered orally, for example, in the form of tablets, dragees, capsules or solutions, or intraperitoneally, intramuscularly, subcutaneously, intraarticularly or intravenously, for example, by means of injection or infusion. It is especially preferred that the application according to the instant invention occurs in such a manner that the active agent is released with delay, that is as a depot.

Unit doses can be administered, for example, 1 to 4 times daily. The exact dose depends on the method of administration and the condition to be treated. Naturally, it can be necessary to vary the dose routinely depending on the age and the weight of the patient and the severity of the condition to be treated.

While the instant invention is discussed in terms of the protein markers, their methods for preparation and uses for diagnostic, therapeutic and drug discovery purposes; the markers may be produced by other known methods and used for other known uses for proteins. For example, once the marker has been identified, it may be produced by extraction from a biological sample or the gene cloned and expressed to produce the protein. Such methodology is well known in the art. Likewise, protein markers have been used for a number of basic research and identification uses such as in pathology, forensics and archeology.

The invention now will be exemplified in the following non-limiting examples.

### EXAMPLE 1

### Sample Selection

Approximately 400 pairs of monozygotic human twins were screened for divergent phenotypic disease states. Serum samples from 158 subjects (79 twin pairs) were selected based on differences in five disease states. The samples were divided into 5 discordant disease groups according to intra-twin clinical trait differences. The quantitative traits were measured to determine the clinical disease area given in the chart below.

| | Clinical Disease | |
|---|---|---|
| Group Number | Area | Quantitative Trait |
| 1 | Obesity | Total Fat Mass, Percentage of Fat |
| 2 | Diabetes | Insulin Resistance |
| 3 | Osteoporosis | SBMD/TOTBMD |
| 4 | Osteo arthritis | OVE |
| 5 | Hypertension/ | CCAI/RCAI |
| | arterial distensibility | |

The samples are whole serum with approximately 70 mg/ml of proteins. The lipids did not significantly interfere with the chromatographic separation. For disease groups 1 and 2, 25 μl of total serum were used. For groups 3, 4 and 5, 50 μl were used.

### EXAMPLE 2

### Serum Fractionation

Protein subtraction columns were prepared to remove common proteins that comprise most of the protein in the sample. For groups 1, 2 and 3, two subtraction columns were prepared and used. The first column (ATH) contained Poros® beads covalently bound to Protein A, Protein G or a mixture of the two, which is then bound to monospecific antisera to certain serum proteins. The antibodies were specific to albumin, transferrin and haptoglobin. The second column contained immobilized wheat germ agglutinin lectin. For groups 4 and 5, the first column had antibody to alpha-1 antitrypsin, albumin, transferrin and haptoglobin with a second column of immobilized Protein A. All antibodies were crosslinked according to the method of Schneider et al., Journal of Biological Chemistry 257:10766-10769 (1982). Approximately 4 ml of immunoaffinity resin were used. Generally, the columns completely removed all of the components, which they specifically bound except for group 3 where a small amount of albumin was carried over.

Samples of about 70 mg/ml protein were used with 25-50 μl being added which corresponds to about 1.7 mg to 3.4 mg of the protein. The samples were loaded into the first HPLC column. Unbound protein fraction from the ATH column was eluted using a 0.5 M ammonium bicarbonate buffer and transferred to the second column. A first unbound protein fraction was eluted with 0.5 M ammonium bicarbonate buffer followed by eluting a bound second protein fraction with 0.5 M N-acetyglucosamine followed by 0.5 M ammonium bicarbonate to wash the protein through. UV 280 nm detected peak areas were observed continually as controls for reproducibility of serum loading and column performance.

In this way, two fractions from each serum protein subtraction experiment were retained. The fractions containing proteins (albumins etc.) removed by the ATH column were released from the matrix by HCL, pH 2.5 or acetic acid, pH 2.5-3 were discarded and the columns equilibrated to be reused for the next sample.

Quantitatively, about half of the serum protein was removed from the sample. The first protein fraction contains non-glycosylated proteins without sialic acid chains and the second protein fraction contains glycosylated proteins. The final protein concentration ranged between 12 and 22 mg/ml in 0.5 M ammonium bicarbonate buffer.

Both collected fractions were collected in 2-4 ml of 0.5 M ammonium bicarbonate buffer and underwent concentration to 100 μl followed by buffer exchange to 4 ml twice in 25 mMol ammonium bicarbonate by ultrafiltration on a membrane unit with an approximately 5,000 dalton molecular weight cut off. About 100 μl were retained and lyophilized. The presence of proteins is visible because both fractions contain molecules (heme, iron, porphyrin etc.) that absorb light in the visible range.

The fractions were resolubilized in 25-50 μl solubilizing solution below. About 2 mg protein is present (70 mg/ml plus solubilizing solution), thus some proteins were not solubilized under the denaturing conditions. About 5-20 μl were loaded onto a gel for each sample.

For Groups 4 and 5, the methods above were repeated with the second column being immobilized protein A. The unbound proteins were recovered. An elution buffer of acetic acid, pH 3-2.5, equilibrated the column.

### EXAMPLE 3

### 2-Dimensional Electrophoresis

Protein aliquots (about 8 μl) of fractionated serum proteins were loaded onto the gels.

The samples were solubilized in 9 M urea, 2% CHAPS, 0.5% dithiothreitol (DTT) and 2% carrier ampholytes, pH 8-10.5.

Ultrapure reagents for polyacrylamide gel preparation were obtained from Bio-Rad (Richmond, CA). Ampholytes, pH 4-8, were from BDH (Poole, UK), ampholytes pH 8-10.5 were from Pharmacia (Uppsala, Sweden) and IGEPAL-630 was obtained from Sigma (St. Louis, Mo.). Deionized water from a high purity water system (Neu-Ion, Inc., Baltimore, Md.) was used. System filters are changed monthly to ensure 18 MΩ purity. Dithiothreitol (DTT) was obtained from Gallard-Schlesinger Industries, Inc. (Carle Place, N.Y.). All chemicals (unless specified) were reagent grade and used without further purification.

Sample proteins were resolved with two-dimensional gel electrophoresis using automated and controlled versions of the 20×25 cm ISO-DALT® 2-D system (Anderson et al., Electrophoresis 12(11) 907-930, 1991). Solubilized samples were applied to each IEF gel, and the gels were run for 25,550 volt-hours using a progressively increasing voltage with a high-voltage programmable power supply. An Angelique™ computer-controlled gradient-casting system (Large Scale Biology Corporation, Rockville, Md.) was used to prepare the second-dimension SDS slab gels. The top 5% of each gel was 11% T acrylamide and the lower 95% of the gel varied linearly from 11% to 19% T for groups 1, 2 and 3. For groups 4 and 5, the top 5% of each gel was 8% T and the lower 95% the gel varied linearly from 8-15% T. The IEF gels were loaded directly onto the slab gels using an equilibration buffer with a blue tracking dye and were held in place with a 1% agarose overlay. Second-dimensional slab gels were run overnight at 160 V in cooled DALT tanks (10° C.) with buffer circulation and were taken out when the tracking dye reached the bottom of the gel for groups 1-3. For groups 4 and 5, the conditions were 2-3 hours at 600 V and 20° C.

For Coomassie blue (CB) staining followed by silver staining for gels for groups 1-3, following SDS electrophoresis, the slab gels were fixed overnight in 1.5 liters/10 gels of 50% ethanol/3% phosphoric acid and then washed three times for 30 min in 1.5 liters/10 gels of cold deionized (DI) water. They were transferred to 1.5 liters/10 gels of 34% methanol/17% ammonium sulfate/3% phosphoric acid for one hour, and after the addition of one gram powdered Coomassie Blue G-250, the gels were stained for three days to achieve equilibrium intensity.

Stained slab gels were scanned and digitized in red light at 133 micron resolution, using an Eikonix 1412 scanner and images were processed using the Kepler® software system as described (Richardson et al., Carciniogenesis 15(2) 325-329, 1994). Coomassie blue gels were destained in 1.5 L of 50% ethanol, 45% deionized water and 5% acetic acid overnight and reswollen in DI water for one hour.

For silver staining (AG), the gels were then clipped onto a gel hanger and processed through the fully automatic Argentron™ silver stainer. The individual steps include agitation for 30 seconds in deionized water, one minute in 0.44 g sodium thiosulfate in 2 L DI water, 10 seconds in deionized water, 30 minutes in 4.6 g silver nitrate in 2 L DI water and 0.78 ml 37% formaldehyde, 10 second DI water wash, 20 minutes in 66 g potassium carbonate, 0.033 g potassium thiosulfate in 2L deionized water with 0.78 ml of 37% formaldehyde. Images are taken at 30 second intervals and the development is stopped with 88 g tris (hydroxymethyl)aminomethane in 2 L deionized water and 44 ml glacial acetic acid.

For groups 4 and 5, the gels were fixed in 1.5 L of 50% ethanol and 3% phosphoric acid in 47% deionized water for 4 hours and then washed in DI water for 1 hour. The gels are clipped into gel hanger and processed as above.

The images were assembled and then processed using the Kepler® software system as described above for the silver stained gels.

### EXAMPLE 4

### Determination of Protein Markers

The Coomassie blue stained gels averaged a few hundred quantifiable protein spots per gel while silver stained gels averaged between one and one-half and two times as many spots per gel. The samples were mixed with rat liver homogenate, a well-characterized sample where a very large number of proteins have been completely identified. From the co-electrophoresis gel, the sample spots were “WARPED” using the method of U.S. Ser. No. 09/643,675 and the “IMPRESS” method of U.S. Ser. No. 09/653,363. The data regarding the protein spots identified is given in the Tables above and Figures.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents, applications and references cited herein are explicitly incorporated by reference in their entirety.

What is claimed is:

1. A method for determining a disease state of a subject comprising;
obtaining a biological sample containing protein from said subject,
measuring levels of protein markers of the disease state in said sample, and
comparing the levels of said markers to the levels of the same markers in a control sample from a subject not having the disease state or a control standard.

2. The method of claim 1 wherein the protein markers are selected from the group consisting of the markers in Tables 1-5.

3. The method of claim 2 wherein the protein markers are selected from the group consisting of the markers for the conditions of obesity, osteoporosis, diabetes, osteoarthritis or hypertension.

4. The method of claim 1 wherein said disease state is selected from the group consisting of obesity, osteoporosis, diabetes, osteoarthritis and hypertension.

5. The method of claim 1 wherein the levels of protein markers determines the relative severity of the disease state.

6. The method of claim 1 further comprising;
measuring levels of individual proteins in a proteome of said biological sample from the subject,
comparing these levels with levels of the same proteins in the proteome from a sample from a control subject or a control standard, and
detecting which proteins are increased or decreased by a statistically significant amount
wherein the proteins so detected are the markers for the disease state.

7. The method of claim 6 wherein the statistically significant amount is determined as a p<0.01.

8. The method of claim 7 wherein p<0.001.

9. The method of claim 6 wherein said disease state is selected from the group consisting of obesity, osteoporosis, diabetes, osteoarthritis and hypertension.

10. The method of claim 6 wherein said proteome is prepared by two-dimensional electrophoresis.

11. A protein marker selected from the proteins of Tables 1-5.

12. A protein marker of claim 11 selected from the proteins are markers for the diseases of obesity, osteoporosis, diabetes, osteoarthritis or hypertension.

13. A binding reagent bound to a detectable label specific for a protein selected from the group consisting of protein markers of claim 11.

14. A binding reagent bound to a detectable label specific for a protein selected from the group consisting of protein markers of claim 11.

15. A method of monitoring efficacy of a therapy for a disease state in a subject comprising;
obtaining a biological sample containing protein from said subject,
measuring levels of protein markers of the disease state in said sample, and
comparing the levels of said markers to the levels of the same markers in the same subject at a previous time.

16. The method of claim 15 wherein the disease state is selected from the group consisting of obesity, osteoporosis, diabetes, osteoarthritis and hypertension.

17. A protein selected from the group consisting of proteins listed in Tables 1-5.

18. A protein according to claim 17 in isolated form.

19. A binding reagent specific for the protein of claim 17.

20. The binding reagent of claim 19 bound to a detectable label.

21. A method for screening candidate compounds biological activity against obesity, osteoporosis, diabetes, osteoarthritis or hypertension comprising;
contacting a candidate compound with a subject having obesity, osteoporosis, diabetes, osteoarthritis or hypertension,
measuring the level of a protein marker of Tables 1-5, and
comparing the level of protein marker to the level of protein marker in a control sample from a subject not having the disease state or a control standard.

22. A pharmaceutical composition comprising;
a modifier of the level of or the activity of a protein marker of Tables 1-5, and
a pharmaceutically acceptable carrier,
wherein said modifier was identified by the process of claim 21.

23. The pharmaceutical composition of claim 22, wherein the modifier is in an effective amount for treating obesity, osteoporosis, diabetes, osteoarthritis or hypertension.

24. A method for treating a disease state comprising;
administering an effective amount of a modifier of the level of or the activity of a protein marker of Tables 1-5, and a pharmaceutically acceptable carrier,
wherein said modifier was identified by the process of claim 21.

25. The method of claim 24, wherein the modifier is in an effective amount for treating obesity, osteoporosis, diabetes, osteoarthritis or hypertension.

26. A method for screening candidate compounds for detection or therapeutic activity against a disease state comprising;
contacting a candidate compound with a protein marker of Tables 1-5,
measuring the activity of said protein marker or the binding of said compound to said protein marker, and
selecting for further development those compounds which affect activity or bind.

27. A method of identifying biological pathways involved in a disease state, comprising;
a) obtaining a biological sample from a subject having obesity, osteoporosis, diabetes, osteoarthritis or hypertension,
b) determining levels of proteins in the proteome in said biological sample,
c) comparing the levels of each protein in said proteome to levels of protein in a control sample from a subject not having the disease state or a control standard,
d) determining which proteins have statistically significantly higher or lower levels in each sample,
e) identifying a plurality of the determined proteins, and
f) deducing which biological pathways are affected based on the identities of said proteins,
wherein said biological pathways contain at least one protein having a statistically significantly higher or lower level in a comparison between the two samples.

28. The method of claim 27, wherein one sample has a combination of two or more protein markers which have statistically significantly higher or lower levels than the same combination of protein markers in the other sample.

29. A standardized two-dimensional electrophoretic distribution of proteins from a biological sample from a subject having obesity, osteoporosis, diabetes, osteoarthritis or hypertension.

30. The standardized two-dimensional electrophoretic distributions of proteins according to claim 29 wherein the biological sample is human serum.

31. The standardized two-dimensional electrophoretic distribution of proteins of claim 29 wherein said subject is being treated with pharmaceuticals indicated for the same conditions.

32. The method according to claim 15, wherein the proteome of the biological sample is measured.

33. The method according to claim 23, wherein the proteome of the biological sample is measured.

34. The method according to claim 26, wherein the disease state is obesity, osteoporosis, diabetes, osteoarthritis or hypertension.

35. A method for determining whether a combination of proteins together form a protein marker of a disease state when the proteins individually are not markers with a desired level of statistical significance, comprising;
determining proteins that are at altered levels in biological samples from a subject having the disease state and controls or biological samples from a subject without the disease state, which proteins are less than the desired level for statistically significant markers by themselves,
selecting two or more of said proteins,
combining the values for two or more of said proteins and determining whether the combination of values is altered in a statistically significant manner,
wherein said combination of proteins results in the desired level of statistically significant differences between biological samples from subjects with the disease state and controls or biological samples from a subject without the disease state.

36. The method of claim 35, wherein said disease state is obesity, osteoporosis, diabetes, osteoarthritis or hypertension.

37. A composition comprising the combination of proteins of claim 36 forming the protein marker.

38. A set of binding reagents, wherein a binding reagent specifically binds to each different protein in the composition of claim 37.

39. A method for finding drug development targets for obesity, osteoporosis, diabetes, osteoarthritis or hypertension comprising;
measuring the level of each protein in a proteome of a biological sample containing protein from a subject having obesity, osteoporosis, diabetes, osteoarthritis or hypertension,
comparing the level of each protein to the level in a control biological sample,
determining which proteins are found in a statistically significant abnormal amount thereby indicating them to be protein markers, and
determining which of the protein markers is involved in the same metabolic pathway as said disease state, thereby indicating these to be drug development targets.

40. Drug development targets determined by the method of claim 39.

41. A binding reagent specific for the drug development targets of claim 39.

42. The binding reagent of claim 41 bound to a detectable label.

43. The drug development targets of claim 40 selected from those of Tables 1-5.

44. The drug development targets of claim 43 for the diseases of obesity, osteoporosis, diabetes, osteoarthritis or hypertension.

45. A method for determining whether a protein is a protein marker of a disease state when the protein is not a statistically significant marker comprising;
a) determining protein markers for a disease state and protein submarkers that have an altered level but are altered to less than a statistically significant amount by themselves, and
b) comparing the level and direction of change of protein markers with the protein submarkers,
wherein protein submarkers that are altered in tandem consistently with protein markers in level and direction or opposite direction are themselves considered protein markers.

46. A protein submarker produced by the method of claim 45.

47. A binding reagent specific for a protein submarker of claim 45.

48. A method for generating an index marker for a particular physiological state comprising;
determining protein markers that differ in a statistically significant manner between biological samples from a subject with a disease state and a control biological sample, which proteins are statistically significant protein markers by themselves,
selecting two or more of said protein markers,
combining the values for two or more of said protein markers and determining whether the combination of values is altered in a manner of greater statistical significance.

49. An index marker determined by the process of claim 48.

50. A method for cloning a gene encoding a protein in Tables 1-5 comprising,
determining at least a partial amino acid sequence of said protein,
deducing a nucleotide sequence for a gene encoding said protein, and
isolating or synthesizing a gene encoding said nucleotide sequence.

51. The gene for a protein in Tables 1-5 produced by the process of claim 50.

52. An antisense compound capable of inhibiting expression of the gene of claim 51.

53. A method for determining whether plural agents act in an additive or synergistic manner comprising;
exposing a subject to a first agent and obtaining a protein containing biological sample thereof,
exposing a subject to a second agent and obtaining a protein containing biological sample thereof,
exposing a subject to a first agent and a second agent and obtaining a protein containing biological sample thereof,
measuring the levels of protein markers in each biological sample,
comparing the changes in levels of protein markers between a subject exposed to a first agent, a subject exposed to a second agent and a subject exposed to a first and second agent and
determining whether the effects of said first agent and said second agent are cumulative or synergistic.

54. A pharmaceutical composition comprising said first agent and said second agent when the effects are more than additive as determined by the method of claim 53.

55. The method of claim 53 wherein said markers are selected from those of Tables 1-5.
